(19) United States
(12) Patent Application Publication
(10) Pub. No.: US 2025/0380709 A1
(43) Pub. Date: Dec. 18, 2025

(54) PROCESSING AID FOR THE PREPARATION OF DOUGHS FOR BAKERY PRODUCTS

(71) Applicant: IL GRANAIO DELLE IDEE S.R.L., Masera' Di Padova (IT)

(72) Inventors: Federico Allamprese Manes Rossi, Albignasego (IT); Angiolella Lombardi, Breganze (IT)

(21) Appl. No.: 18/878,282

(22) PCT Filed: Jul. 6, 2023
(86) PCT No.: PCT/IB2023/056983

§ 371 Date: Dec. 23, 2024

(30) Foreign Application Priority Data
Jul. 7, 2022 (IT) 102022000014398

(51) Int. Cl. A21D 8/04; A23P 10/30

(52) U.S. Cl. CPC A21D 8/045; A21D 8/042; A23P 10/30

(57) ABSTRACT

An aid for the preparation of doughs for baked food products. Such aid comprises a food-grade carrier for baked food products and microcapsules containing viable lactic acid bacteria of at least one species, enclosed in a casing including at least one gellable biopolymer. The microcapsules are dispersed in said food-grade flour and have an average size comprised between 350 μm and 550 μm, preferably between 390 μm and 490 μm, more preferably between 400 μm and 460 μm, and a mechanical compressive strength comprised between 3200 g and 9000 g, preferably between 3400 g and 6000 g, more preferably between 3500 g and 5700 g. A microencapsulation process, a production process and uses of such aid is further provided, as well as to a dough, a preparation process of baked food products wherein such aid is used, and a baked food product obtainable by such preparation process.

### CROSS-REFERENCE TO RELATED APPLICATION DATA

This application is a U.S. National Stage Application of International Application No. PCT/IB2023/056983, filed Jul. 6, 2023, which claims the benefit of and priority to Italian Patent Application No. 102022000014398, filed Jul. 7, 2022 the disclosure of each is incorporated herein by reference in its entirety.

### FIELD OF THE INVENTION

The present invention relates to a processing aid for the preparation of doughs for bakery products, as well as to the related production process and uses.

The present invention also relates to a dough comprising the aforementioned processing aid, a baked food product and a production process using the aforementioned processing aid.

The present invention also relates to a microencapsulation process of viable lactic acid bacteria, resulting in microcapsules used for the production of the aforementioned processing aid.

### BACKGROUND OF THE INVENTION

Lactic acid bacteria are widely used in the food industry. They are responsible for the fermentation of numerous foods such as dairy products, baked goods, fermented meats or vegetables. Thanks to their metabolic activity, lactic acid bacteria are in fact able to transform raw materials and bring particular rheological and sensory features to food.

In the baked food production field, lactic acid bacteria are often used in addition to or instead of yeast in the preparation of doughs. Lactic acid bacteria are contained in the so-called sourdough or sourdough yeast, a mixture of water and flour left to ferment spontaneously in which a complex biological ecosystem develops and is maintained, which varies depending on the environment, the raw materials used and the production method. The dominant microflora of sourdough includes lactic acid bacteria in association with yeasts, generally present in smaller quantities than lactic acid bacteria.

The use of sourdough confers advantageous rheological and shelf-life features to the dough and better organoleptic qualities to the finished products. The fermentation of sugars by the lactic acid bacteria present in the sourdough produces organic acids such as lactic acid and, in the case of obligate heterofermentative lactic acid bacteria, acetic acid, as well as carbon dioxide. Lactic acid bacteria can also produce polysaccharides and, thanks to their enzymatic activities, degrade proteins in flours and produce volatile compounds that confer to the products complex and characteristic aromatic profiles.

The preparation of traditional sourdough, also referred to in the industry as type 1 sourdough, starts with a spontaneous sourdough obtained from a mixture of water and flour left to acidify spontaneously for a longer or shorter time. Fermentation and the subsequent rising of the dough are triggered by lactic acid bacteria naturally present in the flour and the environment. The spontaneous sour dough can then be renewed at regular intervals by subsequent re-mixing with water and flour in doses appropriate to the dough's features.

This type of preparation gives rise to a chaotic and uncontrolled development of various microbial species, not always with positive features, which are spontaneously selected as the acidity of the mixture increases and the oxygen and sugar content decreases during fermentation. In general, unwanted organisms are inactivated, but the variability of the microflora often does not allow its features to be preserved for long, sometimes making the technological performance of natural sourdough yeast unstable and difficult to control. This, combined with the laborious handling, long leavening times and delicate storage of sourdough, means that its use is limited to the home or at most small-scale industrial production.

### RELATED ART

In order to replicate the properties of spontaneously fermented semi-finished products or foodstuffs, such as the aforementioned doughs based on sourdough, or to impart special functional or organoleptic properties to foodstuffs, it is known to technologically incorporate viable lactic acid bacteria into raw materials (e.g. flour, milk, meat, etc.), semi-finished products or foodstuffs, or into additives or processing aids intended for the preparation of such semi-finished products or foodstuffs.

However, the industrial-scale use of lactic acid bacteria presents several critical issues, starting with the need to preserve the viability of the microorganisms and ending with the use of the additive, processing aid, semi-finished product or foodstuff in which these bacteria are dispersed.

To address these issues, microencapsulation technologies are known in the industry to incorporate viable bacterial cells into small capsules of food-grade polymer material.

For example, document KR20160051902A discloses compositions including lactic acid bacteria embedded in alginate beads.

Alginate beads are produced by mixing lactic acid bacteria with an aqueous solution of alginate and adding this solution dropwise to a calcium chloride solution to harden the beads. The resulting beads are dried by freeze-drying.

The document points out that the use of freeze-drying as a method for drying capsules containing lactic acid bacteria impairs the viability of the bacteria, being associated with a survival rate of only 60-70% after drying. To overcome this drawback and increase the survival rate of the bacteria, various cryoprotectants are used, e.g. soy flour, sugars, amino acids, peptides, gelatin, glycerol, sugar alcohols, whey, alginic acid, ascorbic acid, yeast extract, skimmed milk, trehalose, garlic extract.

The Applicant notes that freeze-drying is a complex and expensive drying technique, as well as rather energy inefficient. The process requires subjecting the material to be dried to very low temperatures under high vacuum conditions for several days. These and other critical issues make this technique difficult to scale on an industrial level.

Freeze-drying also has a strong impact on the viability of encapsulated microorganisms. To address this problem, KR20160051902A relies on the use of cryoprotectant additives, which, however, as the document itself acknowledges, change the taste, texture and/or production costs of the food in which the disclosed lactic acid bacteria compositions are used, which may make them unsuitable for the preparation of many foods.

It is also noted that the document does not focus on the structural, organoleptic and nutritional features of the finished products obtained from compositions including microcapsules, features that are particularly important, in the Applicant's opinion, especially when lactic acid bacteria are used in the preparation of baked food products with the intention of reproducing the effects of fermentation promoted by natural sourdough yeast.

### SUMMARY OF THE INVENTION

In view of the problems encountered in the prior art, a general aim of the present invention is to make available a food aid comprising microencapsulated lactic acid bacteria that has a high degree of stability over time, and in particular is capable of maintaining the viability of lactic acid bacteria substantially unaltered for at least six months of storage at room temperature.

More specifically, the Applicant's objective was to provide a food additive comprising capsule structures configured to preserve the viability of microorganisms during the storage period, while at the same time allowing for easy release and rapid dispersion of the microorganisms in the dough when the aid is added to it during mixing.

A further objective of the present invention is to provide a food additive for the preparation of dough for baked food products that is able to give products made with it organoleptic and nutritional qualities, improved shelf-life and structure compared to products made with known processing aids and comparable to those found in products made with natural sourdough yeast.

The Applicant has also set itself the goal of providing a food aid including microcapsules containing lactic acid bacteria that can be obtained by a simplified, more economical process and that does not require the use of protective additives, such as cryoprotectants, during the step of drying the microcapsules.

With these objectives in mind, the inventors perceived and experimentally found that, by providing microcapsules with appropriate chemical-physical, mechanical and geometric properties, their resistance to rupture during storage and subsequent use can be appropriately regulated.

The present invention therefore relates, in a first aspect thereof, to a processing aid for the preparation of dough for baked food products, comprising food grade flour and microcapsules dispersed in food grade flour.

The microcapsules contain viable lactic acid bacteria enclosed in a casing including at least one gellable biopolymer, and have an average size D50 comprised between 350 μm and 550 μm, preferably between 390 μm and 490 μm, more preferably between 400 μm and 460 μm. The microcapsules also have a mechanical compressive strength, measured as detailed in Example 4, comprised between 3200 g and 9000 g, preferably between 3400 g and 6000 g, more preferably between 3500 g and 5700 g.

In accordance with a second aspect of the invention and in the context of the solution to the proposed technical problem illustrated above, the invention further relates to a process for microencapsulating viable lactic acid bacteria in a casing comprising at least one gellable biopolymer.

This process comprises:
a) providing a solution including a gellable biopolymer and viable lactic acid bacteria;
b) feeding drops of said solution of gellable biopolymer and viable lactic acid bacteria into a gelation bath including at least one gelling agent and allowing these drops to gel, and
c) drying the gelled droplets by air drying, obtaining microcapsules containing viable lactic acid bacteria belonging to at least one species, said microcapsules having an average size D50 comprised between 350 μm and 550 μm, preferably between 390 μm and 490 μm, more preferably between 400 μm and 460 μm, and a mechanical compressive strength, measured as detailed in Example 4, comprised between 3200 g and 9000 g, preferably between 3400 g and 6000 g, more preferably between 3500 g and 5700 g.

In accordance with a third aspect of the invention and in the context of the solution to the proposed technical problem illustrated above, the invention further relates to a process for producing an aid for the preparation of baked food products.

This process comprises:
x) providing, by means of a microencapsulation process in accordance with the second aspect of the present invention, microcapsules containing viable lactic acid bacteria belonging to at least one species, enclosed in a casing including at least one gellable biopolymer, said microcapsules having an average size comprised between 350 μm and 550 μm, preferably between 390 μm and 490 μm, more preferably between 400 μm and 460 μm, and a mechanical compressive strength, measured as detailed in Example 4, comprised between 3200 g and 9000 g, preferably between 3400 g and 6000 g, more preferably between 3500 g and 5700 g; and
y) dispersing said microcapsules in a quantity of a food-grade carrier for baked food products.

In accordance with a fourth aspect and in the context of the solution to the proposed technical problem illustrated above, the invention further relates to a dough for baked food products comprising an aid according to the first aspect of the invention, water and optionally further ingredients.

In accordance with a fifth aspect and in the context of the solution to the proposed technical problem outlined above, the invention also relates to a process for preparing a baked food product, comprising:
forming a slurry by mixing the aid according to the first aspect of the invention with water and, optionally, further ingredients such as flour,
optionally, dividing the dough into portions;
optionally, giving the dough or portions of dough a desired shape; and
baking the dough or portions of dough in an oven.

In accordance with a sixth aspect and in the context of the solution to the proposed technical problem illustrated above, the invention also relates to a baked food product obtainable by the preparation process in accordance with the fifth aspect of the invention.

In accordance with a seventh aspect, the invention further relates to the use of a processing aid according to the first aspect in the preparation of doughs for baked food products.

In accordance with a further unclaimed aspect, the invention further relates to the use of a processing aid according to the first aspect of the invention in a straight dough method for producing a baked food product according to the third aspect of the invention.

The Applicant surprisingly found that the processing aid of the invention imparts organoleptic, rheological and textural properties to baked goods made with it, or textures similar to those of products made from sourdough.

Without wishing to be bound herein to any interpretative theory, the inventors believe that the positive effect of improving the organoleptic, rheological and structural properties or texture of baked food products may be attributed to the aforementioned combination of dimensional features and mechanical strength of the microcapsules.

In particular, the inventors believe that the above dimensions and mechanical strength values are the optimal compromise to give the microcapsules the ability to remain intact and preserve lactic acid bacteria in the absence of mechanical stress and in a dry environment, and to open quickly when exposed to the mechanical stresses typical of dough preparation, whether applied manually or mechanically by kneading machines, and to the wet environment of the dough.

The Applicant also considers that the dimensions of the microcapsules obtained according to the microencapsulation process of the invention, which are smaller than those disclosed by the known technique referred to herein, facilitate their dispersion and homogeneous distribution in the doughs during mixing.

The inventors surprisingly experimentally found that the use of an air drying step in the microencapsulation process according to the invention results in microcapsules having the aforementioned advantageous dimensional and mechanical strength features.

The inventors also found that, in addition to resulting in microcapsules containing lactic acid bacteria suitable for imparting improved properties to baked food doughs, the microencapsulation process according to the invention is less complex, less expensive and more easily scalable than known microencapsulation processes such as the one disclosed by KR20160051902A including a freeze-drying step.

The inventors have also experimentally found that the activity of the lactic acid bacteria enclosed in the microcapsules remains substantially unaltered for at least six months of storage of the processing aid. Surprisingly, lactic acid bacteria activity was found to be maintained even when storing the processing aid at room temperature or at the temperatures at which bakery food doughs are generally processed, around 28-30° C., demonstrating the high stability of the processing aid of the invention.

### DETAILED DESCRIPTION OF CURRENTLY PREFERRED EMBODIMENTS OF THE INVENTION

The present invention may include, in one or more of its aspects, one or more of the preferred features outlined below, which can be combined with one another as preferred according to the application requirements.

In the context of this description and subsequent claims, “baked food product” means any savoury or sweet product obtained by total or partial baking of a leavened and/or fermented dough obtained by mixing food-grade flour products with water and any additives or processing aids. By way of non-limiting example, this term may designate products such as bread, pizza, focaccia, biscuits, crackers, rusks, brioches, cakes and baked desserts, bread sticks, taralli.

In this description and in subsequent claims, the terms “straight dough method/process” and “indirect dough method/process” are used in their common meaning in the field of baked food preparation. In particular, “straight dough making method/process” refers to a method/process for preparing a dough for baked food products in which the ingredients are added all together or one after the other in sequence and immediately kneaded. In contrast, the “indirect dough method/process” refers to a method/process in which a pre-dough is made with only part of the ingredients, this dough is left to rest for a predetermined time that can range from a few minutes to a few hours, and then the remaining ingredients are added.

In the context of the present description and in the subsequent claims, “added enzyme” means an enzyme distinct from and additional to enzymes possibly produced exogenously by the metabolism of microorganisms-in particular, lactic acid bacteria-present in the processing aid and/or in the mixture to which such processing aid is added. This added enzyme is added to the processing aid during its production, before the processing aid is used in a dough.

In the context of this description and subsequent claims, the expression “average size D50” used with reference to microcapsules means the percentile diameter relative to 50% of the size distribution of the microcapsules. D50 is defined as the value below which the diameter of 50% of the microcapsules of the size distribution falls.

In the context of the present description and subsequent claims, the expression “average size D10” used with reference to microcapsules means the percentile diameter relative to 10% of the size distribution of the microcapsules. D10 is defined as the value below which the diameter of 10% of the microcapsules of the size distribution falls.

In the context of the present description and subsequent claims, the expression “average size D90” used with reference to microcapsules means the percentile diameter relative to 90% of the size distribution of the microcapsules. D90 is defined as the value below which the diameter of 90% of the microcapsules in the size distribution falls.

In the context of this description and subsequent claims, “effective amount of enzyme” means the smallest amount that enables the enzyme to exert its characteristic effect.

Within the context of the present description and following claims, all the numerical magnitudes indicating quantities, parameters, percentages, and so on are to be considered preceded in every circumstance by the term “about” unless indicated otherwise. Furthermore, all ranges of numerical quantities are to be understood as including extremes, unless otherwise indicated, and include all possible combinations of maximum and minimum numerical values and all possible intermediate ranges, in addition to those specifically indicated below.

Preferably, the microcapsules contain lactic acid bacteria that can be isolated from natural sourdough yeast.

More preferably, the microcapsules contain lactic acid bacteria that can be isolated from traditional sourdough yeast, also known in the industry as traditional or type 1 sourdough.

The Applicant has found that bakery products made with a processing aid comprising the lactic acid bacteria of the invention, which can be isolated from traditional sourdough yeast, exhibit recognisable aromas, fragrances and taste which are attributable, in the Applicant's opinion, to the release by the bacteria of a broad spectrum of characteristic volatile compounds such as acids, alcohols, aldehydes, ketones.

The Applicant further noted that the presence of the lactic acid bacteria of the invention promotes the production, during the fermentation of the dough, of lipolytic enzymes capable of breaking down triglycerides and other lipids releasing glycerol, fatty acids and other molecules having a favourable emulsifying power which contributes to improving the quality and structure of the dough.

The Applicant has also found that, due to metabolic mechanisms that have not yet been fully elucidated, baked goods obtained from a processing aid according to the invention are advantageously subject to a slowing down of the staling process and loss of freshness, and keep longer than baked goods obtained without the use of the processing aid of the invention. In particular, the Applicant found that the shelf-life of baked goods obtained by using the processing aid of the invention, evaluated in terms of the starch retrogradation speed, is 30% or more longer than the shelf-life of similar baked goods obtained without the processing aid of the invention.

Preferably, the lactic acid bacteria contained in the microcapsules belong to one or more species selected from Fructilactobacillus sanfranciscensis, Furfurilactobacillus rossiae, Lactiplantibacillus plantarum, Companilactobacillus alimentarius, Levilactobacillus brevis, Lentilactobacillus buchneri, Latilactobacillus curvatus, Lactobacillus delbrueckii, Limosilactobacillus fermentum, Limosilactobacillus frumenti, Levilactobacillus hammesii, Lactobacillus helveticus, Lentilactobacillus hilgardii, Limosilactobacillus panis, Companilactobacillus paralimentarius, Lacticaseibacillus paracasei, Lactiplantibacillus paraplantarum, Lactiplantibacillus pentosus, Limosilactobacillus pontis, Limosilactobacillus reuteri, Latilactobacillus sakei, Lactococcus lactis, Leuconostoc citreum, Leuconostoc mesenteroides, Pediococcus pentosaceus, Weissella cibaria, and Weissella confusa.

More preferably, the lactic acid bacteria contained in the microcapsules belong to one or more species selected from Fructilactobacillus sanfranciscensis, Furfurilactobacillus rossiae, Lactiplantibacillus plantarum.

In preferred embodiments of the invention, the microcapsules contain a combination of lactic acid bacteria belonging to the species Fructilactobacillus sanfranciscensis, Furfurilactobacillus rossiae, and Lactiplantibacillus plantarum.

In other preferred embodiments of the invention, the processing aid comprises a combination of microcapsules each containing lactic acid bacteria belonging to a species selected from Fructilactobacillus sanfranciscensis, Furfurilactobacillus rossiae, and Lactiplantibacillus plantarum.

In this case, the processing aid preferably comprises a first fraction of microcapsules containing lactic acid bacteria belonging to the species Fructilactobacillus sanfranciscensis.

Preferably, the processing aid also includes a second microcapsule fraction containing lactic acid bacteria belonging to the species Furfurilactobacillus rossiae.

Preferably, the processing aid also comprises a third microcapsule fraction containing lactic acid bacteria belonging to the species Lactiplantibacillus plantarum.

Lactic acid bacteria belonging to the species Fructilactobacillus sanfranciscensis, Furfurilactobacillus rossiae, Lactiplantibacillus plantarum are part of the microbiota of many traditional type 1 sourdoughs. In particular, Fructilactobacillus sanfranciscensis and Lactiplantibacillus plantarum are considered key sourdough species, as their presence is reported in approximately 50% of sourdoughs (Ganzle et al. 2016, [1] ). Also Furfurilactobacillus rossiae is widely distributed in sourdoughs, especially in those produced in Italy (Settanni et al. 2005, [2] ).

Fructilactobacillus sanfranciscensis is an obligate heterofermentative bacterial species that metabolises maltose efficiently and is therefore very well adapted to the sourdough environment. Fructilactobacillus sanfranciscensis produces heterohexopolysaccharides (EPS) that positively influence the volume, texture and general rheological features of baked food products. Fructilactobacillus sanfranciscensis also produces metabolites such as phenylactic acid (PLA) capable of inhibiting fungal growth, and contributes to the definition of the flavour of baked food products through the metabolism of maltose and amino acids. Other technologically relevant features of strains of this species may be the increased bioavailability of minerals and bioactive compounds, as well as the ability to degrade gluten (Zhang et al. 2019, [3] ).

Lactiplantibacillus plantarum is a facultative heterofermentative species capable of producing antifungal compounds that can improve the shelf-life of baked goods (Ventimiglia et al. 2015, [4] ). In sourdough, Lactiplantibacillus plantarum is important for acid generation: it ferments hexoses to lactic acid via the homolactic pathway, and degrades pentoses via the pentose phosphate pathway, producing lactic acid and acetic acid. Some strains of Lactiplantibacillus plantarum also possess extracellular protease activity that can have a positive impact on the rheological and sensory features of products.

Furfurilactobacillus rossiae is an obligate heterofermentative bacterial species that is also highly adapted to the sourdough environment. Strains belonging to this species show interesting technological properties, such as acidifying capacity and peptidase activities (Di Cagno et al. 2007, [5] ).

The Applicant observed that the aforementioned lactobacillus species, and in particular Fructilactobacillus sanfranciscensis and Lactiplantibacillus plantarum, are particularly efficient in releasing a broad spectrum and high quantities of volatile compounds that positively impact the aromatic qualities of baked goods.

Preferably, this processing aid also includes an effective amount of at least one added enzyme.

Preferably, the processing aid comprises an amount of said at least one added enzyme comprised between 0.01 and 5 g/kg of processing aid.

In preferred embodiments, said at least one added enzyme is selected from xylanase, transglutaminase, cellulase, amylase, amyloglucosidase, lipase, phospholipase, asparaginase, oxidase of fungal or bacterial origin or mixtures thereof.

Preferably, said at least one added enzyme is of the hydrolysing type.

Preferably, said at least one added enzyme is non-proteolytic.

More preferably, said at least one added enzyme comprises a xylanase enzyme capable of hydrolysing arabinoxylans, thus producing xylose.

Arabinoxylans are branched side-chain polysaccharides built from pentose sugars—arabinose and xylose—located in the cell walls of the starchy endosperm, in the bran tissues, as well as in the husk of different types of cereals. By breaking the arabinoxylan chains and producing xylose, the specifically selected enzyme xylanase increases the availability of soluble, fermentable sugars in the dough.

This increased availability of sugars positively influences the metabolism of lactic acid bacteria that ferment xylose, in particular lactic acid bacteria with a heterofermentative metabolism. Compared to the fermentation of glucose and maltose (hexose sugars), the fermentation of pentose sugars such as xylose is more advantageous for these microorganisms from an energetic point of view, with positive effects on the acidification rate of the dough and the release of volatile compounds.

Being able to freely exert their metabolic activity with even a competitive advantage over yeasts used in leavening bread, lactic acid bacteria impart high quality sensory, nutritional and structural properties or textures to baked food products comparable to those observed in products made from natural sourdough.

Preferably, said food-grade carrier for baked food products comprises one or more among soft wheat flour, durum wheat flour, maize flour, rice flour, spelt flour, oat flour, pea flour, chickpea flour, soy flour, rye flour, millet flour, linseed flour, sesame seed flour, sunflower seed flour, gluten, maize starch, dried sourdough, brewer's yeast, salt, or mixtures thereof.

Preferably, the additional ingredients used in the preparation of the dough may include one or more of the following: food-grade flour, salt, yeast, sugar, fat and baking additives or aids.

Preferably, the solution of gellable biopolymer and viable lactic acid bacteria is an aqueous solution.

Preferably, the gellable biopolymer of the microcapsule casing is chosen from starch or derivatives thereof, sodium alginate, calcium alginate, potassium alginate, ammonium alginate, gellan gum, xanthan gum, milk protein, gelatine, or mixtures thereof.

In particularly preferred embodiments of the invention, the gellable biopolymer of the microcapsule casing is sodium alginate.

Sodium alginate is a water-soluble polysaccharide isolated from the cell wall of algae, composed of two monomeric units: mannuronic acid and guluronic acid. Having a negative total charge, when exposed to multivalent cations such as calcium ions, the carboxyl groups on the guluronic acid monomers interact with the multivalent cations to form a cross-linked hydrogel. Advantageously, polymerisation can take place at room temperature and no heating step is required, which could negatively impact the viability of the microorganisms to be microencapsulated.

Preferably, the sodium alginate used in accordance with the present invention has a viscosity comprised between 4 and 12 mPa·s when dissolved in a 1% by weight aqueous solution at a temperature of 20° C.

An alginate with the above viscosity values is referred to as low-viscosity alginate. A low-viscosity alginate exhibits high solubility, i.e. it is possible to dissolve high concentrations of alginate-up to 15-20% by weight-in an aqueous solution while maintaining the viscosity of the resulting solution suitable for processing to form microcapsules.

The high concentration of alginate in the encapsulating solution, used in the process of microencapsulation of lactic acid bacteria, means that the alginate wall of the microcapsules that confines the lactic acid bacteria is thicker and stronger, thus offering the micro-organisms greater protection and survival possibilities.

Preferably, the processing aid according to the first aspect of the present invention is obtainable by microencapsulation of viable lactic acid bacteria belonging to at least one species in a casing formed from a solution of gellable biopolymer and viable lactic acid bacteria, said solution comprising sodium alginate having a viscosity within the preferred values indicated above, when dissolved in a 1% by weight aqueous solution at a temperature of 20° C.

Preferably, the concentration of sodium alginate in the solution of gellable biopolymer and viable lactic acid bacteria is comprised between 3% and 20% by weight, more preferably between 3% and 15%, even more preferably between 4% and 7% by weight.

Preferably, the concentration of sodium alginate in the solution of gellable biopolymer and viable lactic acid bacteria is greater than 1% by weight, more preferably greater than 2% by weight, even more preferably greater than 3% by weight, even more preferably greater than 4% by weight, even more preferably greater than 5% by weight, even more preferably greater than 6% by weight, even more preferably greater than 7% by weight, even more preferably greater than 8% by weight, even more preferably greater than 9% by weight, even more preferably greater than 10% by weight.

Preferably, the concentration of sodium alginate in the solution of the gellable biopolymer and viable lactic acid bacteria is less than 20% by weight, more preferably less than 19% by weight, even more preferably less than 18% by weight, even more preferably less than 17% by weight, even more preferably less than 16% by weight, even more preferably less than 15% by weight, even more preferably less than 14% by weight, even more preferably less than 13% by weight, even more preferably less than 12% by weight, even more preferably less than 11% by weight, even more preferably less than 10% by weight, even more preferably less than 9% by weight, even more preferably less than 8% by weight, even more preferably less than 7% by weight, even more preferably less than 6% by weight, even more preferably less than 5% by weight.

Preferably, the processing aid according to the first aspect of the present invention is obtainable by microencapsulation of viable lactic acid bacteria belonging to at least one species in a casing formed from a solution of gellable biopolymer and viable lactic acid bacteria, said solution including sodium alginate at a concentration within the preferred values indicated above.

Experiments carried out by the Applicant have verified that the use of alginate, in particular sodium alginate, having the aforementioned viscosity and concentration values in the solution of gellable biopolymer and lactic acid bacteria enables microcapsules to be obtained having dimensions and mechanical properties within the ranges described above and claimed below.

Preferably, the gelation bath is an aqueous solution and said at least one gelling agent includes one or more bivalent cations, e.g. calcium ions from the dissolution of calcium chloride.

Preferably, said step a) of preparing the solution of gellable biopolymer and viable lactic acid bacteria comprises forming an aqueous solution of the gellable biopolymer, adding the lactic acid bacteria and mixing until homogenisation.

Preferably, the concentration of lactic acid bacteria in the solution of gellable biopolymer and viable lactic acid bacteria is comprised between 0.4% and 25% by weight, more preferably between 0.4% and 20% by weight, even more preferably between 0.5% and 20% by weight.

Preferably, lactic acid bacteria are supplied as fresh concentrated biomass in aqueous suspension, as fresh concentrated biomass stored in frozen form or as biomass in freeze-dried form, which can be stored at room temperature, refrigerated or frozen.

When lactic acid bacteria are supplied as fresh or freeze-dried biomass stored in frozen form, a lactic acid bacteria thawing step is provided.

Preferably, the microcapsules have a relative humidity comprised between 10% and 17%, more preferably between 11% and 16%, even more preferably between 12% and 15.5%.

Preferably, the microcapsules have an average size D10 comprised between 200 μm and 320 μm, more preferably between 230 μm and 300 μm, even more preferably between 260 μm and 280 μm.

Preferably, the microcapsules have an average size D90 comprised between 580 μm and 1000 μm, more preferably between 600 μm and 850 μm, even more preferably between 620 μm and 770 μm.

Preferably, the microcapsules have a bulk density comprised between 0.35 g/ml and 0.55 g/ml, more preferably between 0.4 and 0.5 g/ml, even more preferably between 0.45 and 0.48 g/ml, the bulk density being evaluated on an uncompacted bulk mass of microcapsules, and defined as the ratio of the weight to the volume, including voids, of a reference quantity of microcapsules.

Preferably, the microcapsules have a tapped density comprised between 0.45 g/ml and 0.7 g/ml, more preferably between 0.55 and 0.65 g/ml, even more preferably between 0.58 and 0.6 g/ml, the tapped density being evaluated on a compacted bulk mass of microcapsules, and defined as the ratio between the weight and volume, after compaction performed as detailed in Example 3, of a reference quantity of microcapsules.

Preferably, said step b) of feeding droplets of the gellable biopolymer solution and viable lactic acid bacteria into the gelation bath comprises extruding droplets through a preferably vibrating-type nozzle.

In preferred embodiments, said step c) of air-drying the gelled droplets is carried out in an oven with forced air circulation or convection.

Preferably said forced air circulation oven comprises one or more support trays for the microcapsules, an electric coil configured to heat the air in the inner chamber of the oven and a fan configured to move the hot air in the chamber.

Preferably such a forced air circulation oven comprises an outlet at the back configured to release moisture-laden air and an outlet system for exhaust gases.

In alternative preferred embodiments, said step c) of air-drying the gelled droplets is carried out in a vibrating fluid bed dryer.

Preferably, such a vibrating fluid bed dryer comprises a reservoir containing the bulk mass of microcapsules set into vibration by special vibrating elements, into which a flow of hot air is conveyed through the bulk mass of microcapsules as they are moved by the vibration of the reservoir.

The use of this method results in particularly gentle and uniform drying of the microcapsules. Further features of the invention will be illustrated in the following examples.

### Example 1

### Production of Air-Dried Microcapsules

Lactic acid bacteria belonging to bacterial strains of the species Fructilactobacillus sanfranciscensis (SF), Lactiplantibacillus plantarum (PL), and Furfurilactobacillus rossiae (RO) stored in the form of frozen freeze-dried biomass were thawed by immersing the respective tubes in a bath at room temperature.

After thawing, 2.5 g of biomass of each strain was suspended in 200 g of sterile deionised water under agitation, controlling the pH within the range 4.5-5.2.

The bacterial suspension of each of the three Lactobacillus species identified above was then added to an aqueous encapsulating solution containing 7% by weight of sodium alginate extracted from brown algae (BR-L alginate) having a viscosity comprised between 4 and 12 mPa·s when dissolved at 1% by weight in an aqueous solution at a temperature of 20° C.

Solutions of gellable biopolymer and viable lactic acid bacteria were then obtained for subsequent droplet extrusion, the quantities of which are shown in Table 1 below.

TABLE 1
Composition of gellable biopolymer solution and viable
lactic acid bacteria for subsequent extrusion

| | | |
|---|---|---|
| | | Percentage |
| Material | Quantity | by weight |

| | | | |
|---|---|---|---|
| Lactic bacteria | 2.5 | g | 0.5% |
| BR-L alginate | 20 | g | 4% |
| Sterile deionised water | 477.5 | g | 95.5% |
| Total solution | 500 | g | 100% |

Table 2 below shows the pH and viscosity values of the resulting solutions of gellable biopolymer and viable lactic acid bacteria obtained for each bacterial strain.

TABLE 2
pH and viscosity of solutions of gellable
biopolymer and viable lactic acid bacteria

| Solution of gellable biopolymer | | |
|---|---|---|
| and viable lactic acid bacteria | pH | Viscosity |
| Solution with lactic acid bacteria | 6.2 | 59 mPa · s at 25.5° C. |
| SF | | |
| Solution with lactic acid bacteria PL | 6.5 | 65 mPa · s at 24° C. |
| Solution with lactic acid bacteria RO | 6 | 57 mPa · s at 29.6° C. |

Each solution of gellable biopolymer and viable lactic acid bacteria thus obtained was first sieved through an 80 μm sieve to remove any undesirable aggregates and then extruded into droplets using a vibrating nozzle with an outlet diameter of 350 μm (500 Hz, 2750 mV, 150 mBar), dropping the droplets into a gelation bath containing divalent ions from calcium chloride and, optionally, polyethylene glycol of an average molecular weight of 1500 in the quantities specified in Table 3.

TABLE 3
Composition of the gelation bath

| | | |
|---|---|---|
| | | Percentage |
| Material | Quantity | by weight |

| | | | |
|---|---|---|---|
| CaCl₂ | 100 | g | 2% |
| PEG 1500 (optional) | 50 | g | 1% |
| Sterile deionised water | 4850 | g | 97% |
| Total gelation bath | 5000 | g | 100% |

The droplets were left in the gelation bath, resulting in gelled droplets with an average size comprised between 800 μm and 1000 μm.

The resulting gelled drops were washed with a 0.85% NaCl solution and then treated with SiO₂ anti-caking agent.

Finally, the gelled drops were dried in a forced-circulation drying oven by setting an air temperature of 30° C., until the relative humidity of the microcapsules reached between 12% and 15.5%, resulting in a combination of microcapsules each containing a species of lactic acid bacteria selected from Fructilactobacillus sanfranciscensis, Lactiplantibacillus plantarum and Furfurilactobacillus rossiae.

Similar results in terms of the features of the gellable biopolymer and lactic acid bacteria solution (Tables 1 and 2) were obtained by mixing the three aforementioned strains of lactic acid bacteria, during the formation of the gellable biopolymer and lactic acid bacteria solution, to obtain microcapsules each containing a combination of the three strains at the end of the process.

### Example 2 (Comparative)

### Production of Dried Microcapsules by Freeze-Drying

Lactic acid bacteria belonging to bacterial strains of the species Fructilactobacillus sanfranciscensis (SF), Lactiplantibacillus plantarum (PL), and Furfurilactobacillus rossiae (RO) stored in the form of frozen freeze-dried biomass were thawed by immersing the respective tubes in a bath at room temperature.

After thawing, 2.5 g of biomass of each strain was suspended in 200 g of sterile deionised water under agitation, controlling the pH within the range 4.5-5.2.

The bacterial suspension was then added to an aqueous encapsulating solution containing 7% by weight of sodium alginate extracted from brown algae (BR-L alginate) having a viscosity comprised between 4 and 12 mPa·s when dissolved at 1% by weight in an aqueous solution at a temperature of 20° C.

A solution of gellable biopolymer and viable lactic acid bacteria was then obtained for subsequent droplet extrusion, having the same composition as shown in Table 1 above.

The pH and viscosity values of the resulting solutions of gellable biopolymer and viable lactic acid bacteria obtained for each bacterial strain are the same as those provided in Table 2 above.

Each solution of gellable biopolymer and viable lactic acid bacteria thus obtained was first sieved through an 80 μm sieve to remove any undesirable aggregates and then extruded into droplets using a vibrating nozzle with an outlet diameter of 350 μm (500 Hz, 2750 mV, 150 mBar), dropping the droplets into a gelation bath containing divalent ions from calcium chloride and, optionally, polyethylene glycol of an average molecular weight of 1500 in the quantities specified in Table 3 above.

The droplets were left in the gelation bath until completely gelled, resulting in gelled droplets with an average size of approximately 800 μm to 1000 μm.

The resulting gelled drops were washed with a 0.85% NaCl solution, and frozen in liquid nitrogen, and stored at −40° C. before freeze-drying.

The gelled drops were then freeze-dried at −7° C. for 74 h, resulting in a combination of microcapsules each containing lactic acid bacteria belonging to a species selected from Fructilactobacillus sanfranciscensis, Lactiplantibacillus plantarum and Furfurilactobacillus rossiae.

Similar results in terms of the features of the gellable biopolymer and lactic acid bacteria solution (Tables 1 and 2) were obtained by mixing the three aforementioned strains of lactic acid bacteria, during the formation of the gellable biopolymer and lactic acid bacteria solution, to obtain microcapsules each containing a combination of the three strains at the end of the process.

### Example 3

### Characterisation of Microcapsules Containing Lactic Acid Bacteria

The microcapsules containing lactic acid bacteria obtained according to Examples 1 and 2 were characterised by assessing their size distribution, bulk density (evaluated on an uncompacted bulk mass of microcapsules), tapped density (evaluated on a compacted bulk mass of microcapsules), pH and humidity.

The results of this characterisation are shown in the following Table 4.

TABLE 4
Characterisation of microcapsules containing lactic acid bacteria

| | Value | | |
|---|---|---|---|
| | | Microcapsules | |
| | | dried with | |
| | Air-dried | freeze-drying | |
| | microcapsules | (Example 2 | Measurement |
| Parameter | (Example 1) | comp.) | method |
| Average size | 402-458 | 633-643 | Dynamic image |
| D50 [μm] | | | analysis and |
| | | | EQPC method (*) |
| Size | 0.89-1.07 | 0.41-0.47 | Dynamic image |
| distribution | | | analysis and |
| amplitude | | | EQPC method (*) |
| Bulk density | 0.455-0.471 | 0.094-0.098 | USP Method 2 |
| [g/ml] | | (central value | (Sotax) (**) |
| | | 0.096) | |
| Tapped density | 0.589-0.595 | 0.103-0.107 | USP2 method |
| [g/ml] | | | (Sotax) (**) |
| pH | 8.3-8.5 | 7.3-7.5 | Potentiometric |
| | | | measurement on |
| | | | 10% by weight |
| | | | solution |
| Moisture (%) | 12.5-15.1 | 6.2-6.3 | Karl Fischer |
| | | | Method |

(*) The microcapsules were dispersed in free fall with a GRADIS dry dispersal unit (Sympatec), and images of the falling particles were acquired with a high-resolution QICPIC camera (Sympatec) at 80 Hz.
D10 is the value below which the diameter of 10% of the microcapsules of the size distribution falls;
D50 is the value below which the diameter of 50% of the microcapsules of the size distribution falls; and
D90 is the value below which the diameter of 10% of the microcapsules of the size distribution falls.
(**) The bulk and tapped density of the microcapsules was determined with a TD1 tester according to the USP2 method (Sotax). A quantity of microcapsules (about 90 ml) was weighed into a cylinder and the volume was checked to determine the bulk density.

The width of the drop shaft exit slot was set at 40 mm. The M6 measurement interval (2.8 μm-5632 μm) was used for all measurements.

By analysing the images using software, a measurement of the projected area of each microcapsule was extracted. For each projected area extracted, the diameter of the circle with an area equivalent to the projected area was then derived (EQPC method). The diameters thus obtained were used to assess the volumetric size distribution of the microcapsules.

The amplitude of the size distribution was calculated using the following formula:

$$Amplitude = ( D 90 - D 10 ) / D 50$$

in which, as defined above:

To determine the tapped density, the cylinder was raised to 3 mm and dropped under its own weight at a frequency of 250 strokes per minute for a total of 1250 strokes, in order to compact the microcapsules, eliminating voids. The tapped density was then calculated using the final volume evaluated after compaction.

The parameter values in Table 4 show that air-dried microcapsules are smaller and denser, with reference to a bulk mass of microcapsules, than freeze-dried microcapsules. The applicant believes that the smaller size is related to better dispersibility of the microcapsules in the dough during processing. In the Applicant's opinion, the higher density is instead related to the better mechanical properties of the microcapsules (see Example 4 below), which gives the microcapsules greater storage stability.

In addition to the parameters shown in Table 4, the hygroscopic properties of microcapsules containing lactic acid bacteria belonging to each of the three selected species were compared (Fructilactobacillus sanfranciscensis (SF), Lactiplantibacillus plantarum (PL), and Furfurilactobacillus rossiae (RO)), obtained according to Examples 1 and 2.

The hygroscopic behaviour of the samples was quantified using Dynamic Vapor Sorption (DVS) (SPSx-1μ High Load, ProUmid, Germany).

A gradual absorption cycle was applied (from 0% to 90% relative humidity) with a 10% increment for each relative humidity phase. The transition condition to the next phase was determined by an equilibrium condition with 0.01% change in mass for 60 min or a maximum of 48 hours for each phase. The temperature was set to 25° C.

The test results for the hygroscopic behaviour of the microcapsules are shown in Table 5 below.

TABLE 5
Hygroscopicity tests on microcapsules

| | | Microcapsules dried |
|---|---|---|
| Relative | Air-dried | with freeze-drying |
| humidity | microcapsules | (Example 2: |
| phase (%) | (Example 1) | comparative) |
| 0 | 0.0 | 0.0 |
| 10 | 4.6 | 7.3 |
| 20 | 7.1 | 10.9 |
| 30 | 9.4 | 14.2 |
| 40 | 12.1 | 17.9 |
| 50 | 14.8 | 22.3 |
| 60 | 17.8 | 28.8 |
| 70 | 21.2 | 47.3 |
| 80 | 25.4 | 72.6 |
| 90 | 32.0 | N/A |

The data clearly show that freeze-dried microcapsules have a higher mass absorption than air-dried microcapsules at each relative humidity step.

For freeze-dried microcapsules, a strong increase in mass absorption is observed, especially from 70% relative humidity. No reference point at 90% relative humidity could be determined for the freeze-dried microcapsules, as the sample dissolved at such high humidity.

The much more limited hygroscopic character of air-dried microspheres compared to freeze-dried ones suggests a lower sensitivity to humid environments and thus greater storage stability.

### Example 4

### Compression Test of Microcapsules Containing Lactic Acid Bacteria

The compressive strength of microcapsules containing lactic acid bacteria belonging to each of the three selected species (Fructilactobacillus sanfranciscensis (SF), Lactiplantibacillus plantarum (PL), and Furfurilactobacillus rossiae (RO)), obtained according to Examples 1 and 2, was measured using a texture analyser (Perten TVT 6700 Texture Analyzer, with compression probe no. 670159 with a diameter of 21 mm) according to the following process.

A volume of microcapsules was placed in a cylindrical stainless steel container with an internal diameter of 40 mm and a height of 20 mm, so that the filling level of the microcapsules reached 15 mm.

The compression probe was positioned 5 mm above the maximum level of the microcapsule sample.

The vertical descent speed of the probe was set at 1.7 mm/s and a minimum force of 5g was set for the start of the measurement. The downward movement of the probe was stopped upon reaching 40% of the initial height measured at the start of the measurement.

A force-time curve was obtained for each recipe and the maximum force peaks were calculated. For each sample of microcapsules, the compressive strength was then evaluated as the peak force recorded in these temporal force trends.

The results of the compressive strength measurement are shown in Table 6.

TABLE 6
Compression test

| | Compressive |
|---|---|
| Microcapsules | strength [g] |
| Air-dried (Example 1) | 3500-5700 |
| Dried by freeze-drying | 140-160 |
| (Example 2: comparative) | |

The strength measurements show that the dried microcapsules exhibit compressive strength values that are an order of magnitude higher than those dried by freeze-drying. The inventors believe that this is somehow related to the smaller size and higher density observed in air-dried microcapsules (see Example 3 above).

### Example 5 (Invention)

### Production of a Processing Aid Comprising Air-Dried Microcapsules

A combination of microcapsules produced according to Example 1, air-dried and each containing lactic acid bacteria belonging to a species selected from Fructilactobacillus sanfranciscensis (SF), Lactiplantibacillus plantarum (PL), and Furfurilactobacillus rossiae (RO), were mixed with food-grade flour and xylanase enzyme in the quantities indicated in Table 7 below.

TABLE 7
Processing aid with microcapsules containing lactic acid bacteria

| Microcapsules [g] | | | | |
|---|---|---|---|---|
| Lactic | Lactic | Lactic | | |
| bacteria | bacteria | bacteria | Food-grade | Added |
| SF | PL | RO | flour [g] | xylanase [g] |
| 9-13 | 9-13 | 9-13 | 3000 | 0.24 |

The added xylanase is a commercial xylanase (Panzea®, Novozymes) with a manufacturer-declared activity of 235 NXU/g of enzyme (NXU=New Xylanase Unit, a unit of measurement of enzyme activity established internally by the manufacturer).

The food-grade flour used is type “0” wheat flour with the features listed in Tables 8 and 9 below.

TABLE 8
Chemical and physical parameters of food-grade flour

| Parameter | Value | Measurement method |
|---|---|---|
| Humidity [%] | ≤15.5 | D.M. 27 May 1985 |
| | | Determination by oven drying |
| | | and validation of the |
| | | supporting NIR method |
| Ash [% d.m.] | ≤0.65 | ISO 2171 |
| (percentage on dry matter) | | Mould analysis and residual |
| | | weight determination |
| Protein [% d.m.] | ≥11 | NIR infrared, validated by |
| | | official method |
| Fall number [s] | ≥280 | ISO 3093 |
| Dry gluten [% d.m.] | ≥10 | ICC No. 137 |
| | | Mechanical gluten |
| | | determination |
| Enzyme activity | 300-600 | Amilograph ® Brabender |
| α-amylasic | | ICC No. 126/1 |
| [Amylographic units] | | AACC No. 22-10 |
| | | ISO 7973 |

TABLE 9
Rheological parameters food-grade flour

| Parameter | Value | Measurement method |
|---|---|---|
| W [J] | 270 ± 20 × 10⁻⁴ | ISO 5530/4 |
| | | Alveograph - Chopin |
| | | Technologies |
| p/l [ ] | 0.6 ± 1 | ISO 5530/4 |
| | | Alveograph - Chopin |
| | | Technologies |
| Absorption [%] | ≥56 | Farinograph ® Brabender |
| | | ICC No. 115 |
| Development | >1.5 | Farinograph ® Brabender |
| time [min] | | ICC No. 115 |
| Stability [min] | >5 | Farinograph ® Brabender |
| | | ICC No. 115 |
| Fall index 10′ | <70 | Farinograph ® Brabender |
| [Pharinographic | | ICC No. 115 |
| units] | | |

### Example 6 (Comparative)

### Production of a Processing Aid Including Microcapsules Dried by Freeze-Drying

Microcapsules produced according to Example 1, dried by freeze-drying and each containing lactic acid bacteria belonging to a species selected from Fructilactobacillus sanfranciscensis (SF), Lactiplantibacillus plantarum (PL), and Furfurilactobacillus rossiae (RO), were mixed with food-grade flour and xylanase enzyme powder, in the quantities indicated in Table 7 above.

As in the previous example, the food-grade flour used is type “0” wheat flour with the features listed in Table 8 above.

### Example 7

### Bread Preparation

Eight doughs were prepared using a straight dough method, mixing the ingredients in a single step.

The recipes for the 8 doughs, quantities of ingredients and further details are provided in Table 10 below.

TABLE 10
Doughs

| | | Microcapsules | | | | | | | | | | | | |
|---|---|---|---|---|---|---|---|---|---|---|---|---|---|---|
| | | containing lactic | | | Microcapsules | | | | | | | | | |
| | | acid bacteria | | | containing air- | | | Non- | | | | | | |
| | Food- | dried by freeze- | | | dried lactic acid | | | microencapsulated | | | | | | |
| | grade | drying | | | bacteria | | | lactic acid bacteria | | | Added | | | |
| Dough | flour | [g] | | | [g] | | | [g] | | | xylanase | Yeast | Salt | Water |
| No. | [g] | SF | SF | RO | SF | PL | RO | SF | PL | RO | [g] (*) | [g] | [g] | [g] |
| 1 | 3000 | — | — | — | — | — | — | — | — | — | — | 39 | 60 | 2250 |
| (compar.) | | | | | | | | | | | | | | |
| 2 | 3000 | — | — | — | — | — | — | — | — | — | 0.24 | 39 | 60 | 2250 |
| (compar.) | | | | | | | | | | | | | | |
| 3 | 3000 | — | — | — | — | — | — | 9-13 | 9-13 | 9-13 | — | 39 | 60 | 2250 |
| (compar.) | | | | | | | | | | | | | | |
| 4 | 3000 | — | — | — | — | — | — | 9-13 | 9-13 | 9-13 | 0.24 | 39 | 60 | 2250 |
| (compar.) | | | | | | | | | | | | | | |
| 5 | 3000 | 9-13 | 9-13 | 9-13 | — | — | — | — | — | — | — | 39 | 60 | 2250 |
| (compar.) | | | | | | | | | | | | | | |
| 6 | 3000 | 9-13 | 9-13 | 9-13 | — | — | — | — | — | — | 0.24 | 39 | 60 | 2250 |
| (compar.) | | | | | | | | | | | | | | |
| 7 | 3000 | — | — | — | 9-13 | 9-13 | 9-13 | — | — | — | — | 39 | 60 | 2250 |
| (inven.) | | | | | | | | | | | | | | |
| 8 | 3000 | — | — | — | 9-13 | 9-13 | 9-13 | — | — | — | 0.24 | 39 | 60 | 2250 |
| (inven.) | | | | | | | | | | | | | | |

The added xylanase is a commercial xylanase (Panzea®, Novozymes) with a manufacturer-declared activity of 235 NXU/g of enzyme (NXU=New Xylanase Unit, a unit of measurement of enzyme activity established internally by the manufacturer).

The food-grade flour used in all eight doughs is type “0” wheat flour with the features listed in Table 8 above. Brewer's yeast was used in all the doughs.

Doughs 5 and 6 were made using a processing aid as in comparative Example 6, with and without the addition of xylanase enzyme, respectively.

Doughs 7 and 8 were made using a processing aid obtained according to Example 5 in accordance with the invention, with and without the addition of xylanase enzyme, respectively.

Doughs 1 and 2 (comparative) do not include lactic acid bacteria. Doughs 3 and 4 (comparative) include lactic acid bacteria in freeze-dried, non-microencapsulated form.

For each dough, 80% water and all remaining ingredients except salt were mixed in a spiral mixer (VMI BERTO ITALIA SRL model 80 MAG 2V) at speed 1 for about 5 minutes and at speed 2 for about 10 minutes. As soon as the dough became smooth, the remaining water and salt were added. The dough was then left to rest at 26° C. for 45 minutes. After this step, the dough was divided into 100 g portions that were left to rise for 60 minutes at 28° C. and 80% residual moisture.

The portions were then baked for 18 minutes in an electric deck oven (AGIV FORNI model 306-E0) at 210° C. Once baked, the resulting loaves were cooled to room temperature before being evaluated by visual, sensory and texture analysis. For each type of dough, 40 loaves were produced. The loaf obtained with these recipes and this process is called “zoccoletto”, characterised by a marked porosity of the soft part and a very crisp, golden and fragrant crust.

### Example 8

### Loaf Height

The height of the loaves was assessed using a texture analyser (Perten TVT 6700 Texture Analyzer).

Each loaf was placed in the centre of the graduated plate of the texture analyser and the height was measured, on a cm scale, at the point of maximum volume reached. The loaves were also weighed. Average height and weight values were then calculated for the 40 loaves produced according to each recipe.

The average weight and height values of the loaves are shown in Table 11.

TABLE 11
Average loaf height

| | Average | Average |
|---|---|---|
| Dough | loaf weight | loaf height |
| No. | [g] | [cm] |
| 1 (compar.) | 72 | 4.3 |
| 2 (compar.) | 78 | 5.9 |
| 3 (compar.) | 77 | 5.7 |
| 4 (compar.) | 78 | 6.2 |
| 5 (compar.) | 78 | 5.8 |
| 6 (compar.) | 80 | 6.0 |
| 7 (inven.) | 77 | 6.1 |
| 8 (inven.) | 76 | 6.7 |

The weights of the loaves were very similar, with the exception of the loaves obtained from dough 1 in which lactic acid bacteria and xylanase were not used, which was lighter on average.

The average loaf height increases significantly in recipes containing added xylanase combined with lactic acid bacteria. The loaves produced from dough 8, including microcapsules with air-dried lactic acid bacteria (produced according to Example 2) in combination with xylanase enzyme were found to have the highest average height.

The height of the loaf is a relevant aspect in the perception of bread quality, since for the same weight, a greater height and thus a larger loaf volume are associated with a better bread texture.

### Example 9

### Loaf Crust Texture

The crust texture of the loaves of bread was assessed using a texture analyser (Perten TVT 6700 Texture Analyzer).

Each loaf was placed on the analyser measuring table and centred under the analyser probe.

A 25 mm diameter compression probe was applied. The vertical descent speed of the probe was set to 1.7 mm/s. The downward movement of the probe was stopped upon reaching 40% of the initial height measured at the start of the measurement.

A force-time curve was obtained for each loaf of bread analysed. From the curves obtained, the average maximum force peaks were calculated for each type of loaf.

The average peak force values for each type of loaf are shown in Table 12.

TABLE 12
Crust texture

| | Peak force |
|---|---|
| Dough loaves | value (average) |
| No. | [g] |
| 1 (compar.) | 977 |
| 2 (compar.) | 2003 |
| 3 (compar.) | 2194 |
| 4 (compar.) | 3042 |
| 5 (compar.) | 1758 |
| 6 (compar.) | 2086 |
| 7 (inven.) | 2614 |
| 8 (inven.) | 3306 |

The loaf obtained by dough 1 has a not very crispy crust. The addition of xylanase (dough 2) leads to an improvement in crispness. The addition of the non-microencapsulated lactic acid bacteria (doughs 3, 5, 7) induces an improvement in terms of crispness compared to loaf 1, but less so compared to the loaves with enzyme alone (dough 2).

The observed effect is synergistic in that the simultaneous presence of lactic acid bacteria and xylanase significantly increases the crispness of the crust (e.g. doughs 2, 4, 6, 8 compared to doughs 1, 3, 5, 7). The loaves produced from dough 8 show the best results in terms of the peak force required to crumble the crust. This behaviour is typical of a particularly crispy crust.

### Example 10

### Sensory Analysis

The loaves were evaluated on the basis of colour, texture, aroma and taste by a panel of six judges trained to recognise bread quality parameters.

For each type of loaf, the judges assigned a score on a 9-point hedonic scale taking into account sensory features of the different breads such as crust colour, crust cracks, volume increase, soft part porosity, aroma of freshly baked bread, toasted aroma of the crust, lactic notes, acidity, overall taste, sweetness, softness of the soft part during chewing, crunchiness of the crust.

The results of the sensory analysis are shown in Table 13. For each sensory parameter and each loaf of bread, the table shows the average of the scores awarded by each judge. The last column shows the overall average, for each loaf, of all the scores awarded by the judges in relation to all sensory parameters.

TABLE 13
Sensory analysis

| Loaves | VISUAL TEST | | | | OLFACTORY TEST | | | |
|---|---|---|---|---|---|---|---|---|
| from | CRUST | CRUST | | SOFT PART | | TOASTED | TOTAL | |
| dough No | COLOUR | RUPTURES | DEVELOPMENT | POROSITY | FRAGRANCE | ON THE CRUST | LACTIC | ACID |
| 1 | 3.2 | 3.3 | 3.9 | 4.9 | 5.9 | 4.5 | 1.5 | 3.1 |
| (compar.) | | | | | | | | |
| 2 | 7.5 | 7.5 | 7.5 | 7.1 | 6.9 | 6.5 | 4 | 4.1 |
| (compar.) | | | | | | | | |
| 3 | 6.8 | 5.1 | 7.3 | 6.9 | 5.9 | 6 | 7.5 | 4.8 |
| (compar.) | | | | | | | | |
| 4 | 7.7 | 7.7 | 8 | 7.5 | 7.7 | 7.8 | 7.7 | 4.9 |
| (compar.) | | | | | | | | |
| 5 | 7.2 | 7.4 | 7.3 | 6.9 | 6.1 | 6.3 | 8 | 4.5 |
| (compar.) | | | | | | | | |
| 6 | 7.6 | 7.9 | 7.7 | 7.1 | 7.5 | 7.8 | 8.1 | 4.7 |
| (compar.) | | | | | | | | |
| 7 | 7.5 | 7.5 | 7.8 | 7.3 | 7.3 | 7 | 8.6 | 5.1 |
| (inven.) | | | | | | | | |
| 8 | 9.2 | 9.3 | 9.5 | 8.8 | 9.3 | 9.5 | 9.8 | 5.5 |
| (inven.) | | | | | | | | |

| | | | STRUCTURAL TEST | | |
|---|---|---|---|---|---|
| Loaves | | | CHEWING | | AVERAGE |
| from | TASTE | | SOFTNESS | CRUST | SCORE |
| dough No | TOTAL | SWEETNESS | OF SOFT PART | CRUNCHINESS | AWARDED |
| 1 | 4.5 | 3.6 | 4.2 | 2.9 | 3.8 |
| (compar.) | | | | | |
| 2 | 5 | 5.2 | 7 | 7.7 | 6.3 |
| (compar.) | | | | | |
| 3 | 4.9 | 6.9 | 6.3 | 7 | 6.3 |
| (compar.) | | | | | |
| 4 | 6.5 | 7.9 | 7.3 | 8.5 | 7.4 |
| (compar.) | | | | | |
| 5 | 5.5 | 7.1 | 7 | 7.3 | 6.7 |
| (compar.) | | | | | |
| 6 | 7.5 | 7.5 | 7.5 | 9.2 | 7.5 |
| (compar.) | | | | | |
| 7 | 7.2 | 6.8 | 7.1 | 7.5 | 7.2 |
| (inven.) | | | | | |
| 8 | 9 | 8.7 | 8.9 | 9.8 | 8.9 |
| (inven.) | | | | | |

The loaves obtained with the addition of lactic acid bacteria showed a flavour profile in terms of features that are known to be quite relevant for consumer acceptability, such as the appearance of the loaf and the colour of the crust.

The loaves produced from dough 1, which did not include lactic acid bacteria or added enzymes, showed little increase in volume. The crust was pale and not very crispy. Moreover, the taste was neither enveloping nor fragrant.

A good increase in volume and a fair improvement in crust colour was evident for the dough containing only xylanase. An increase in the lactic aromatic component alone is highlighted in loaves 3, 5, 7 containing lactic acid bacteria without added xylanase enzyme.

The loaves obtained from the dough 8 containing microcapsules containing lactic acid bacteria belonging to the species Fructilactobacillus sanfranciscensis (SF), Lactiplantibacillus plantarum (PL), and Furfurilactobacillus rossiae (RO) and air-dried, with the addition of xylanase enzyme, appear to be the most interesting in terms of development, height and crust features. The increase in volume is related to the presence of porous soft part with ample porosity, the crust is crispy, amber-coloured and with long, wide cracks on the surface. The bread is very fragrant, the flavour and aroma are intense with lactic notes.

Naturally, those skilled in the art may make further modifications and variants to the above-described invention with the purpose of meeting specific and contingent application needs, variants and modifications in any case falling within the scope of protection as defined by the successive claims.

### REFERENCES

[1] Gänzle et al., Composition and function of sourdough microbiota: From ecological theory to bread quality, Int J Food Microbiol., 19;239:19-25, 10.1016/j.ijfoodmicro.2016.05.004., 2016.
[2] Settanni et al., In situ activity of a bacteriocin-producing Lactococcus lactis strain. Influence on the interactions between lactic acid bacteria during sourdough fermentation, J Appl Microbiol. 2005;99(3):670-81, 10.1111/j. 1365-2672.2005.02647.x, 2005.
[3] Zhang et al., Prevalence, genetic diversity and technological functions of Lactobacillus sanfranciscensis in sourdough: A review, Comprehensive Reviews in Food Science and Food Safety, 18. 10.1111 (1541) 4337.12459-2019.
[4] Ventimiglia et al, Codominance of Lactobacillus plantarum and obligate heterofermentative lactic acid bacteria during sourdough fermentation, Food Microbiology, 51, 10.1016/j.fm.2015.04.011, 2015.
[5] Di Cagno et al., Genotypic and phenotypic diversity of Lactobacillus rossiae strains isolated from sourdough, Journal of applied microbiology, 103, 821-35, 10.1111/j. 1365-2672.2007.03389.x, 2007.

1-28. (canceled)

29. An aid for the preparation of doughs for baked food products, comprising a food-grade carrier for baked food products and microcapsules containing viable lactic acid bacteria belonging to at least one species, enclosed in a casing including at least one gellable biopolymer,
wherein said microcapsules are dispersed in a food-grade flour and have an average size D50 equal to or higher than 350 μm and equal to or lower than 550 μm and a mechanical compressive strength, measured as set forth in the present description, equal to or higher than 3200 g and equal to or lower than 9000 g.

30. The aid according to claim 29, wherein the lactic acid bacteria in the microcapsules are isolated from natural sourdough yeast type 1.

31. The aid according to claim 29, wherein the lactic acid bacteria contained in the microcapsules belong to one or more species selected from Fructilactobacillus sanfranciscensis, Furfurilactobacillus rossiae, Lactiplantibacillus plantarum, Companilactobacillus alimentarius, Levilactobacillus brevis, Lentilactobacillus buchneri, Latilactobacillus curvatus, Lactobacillus delbrueckii, Limosilactobacillus fermentum, Limosilactobacillus frumenti, Levilactobacillus hammesii, Lactobacillus helveticus, Lentilactobacillus hilgardii, Limosilactobacillus panis, Companilactobacillus paralimentarius, Lacticaseibacillus paracasei, Lactiplantibacillus paraplantarum, Lactiplantibacillus pentosus, Limosilactobacillus pontis, Limosilactobacillus reuteri, Latilactobacillus sakei, Lactococcus lactis, Leuconostoc citreum, Leuconostoc mesenteroides, Pediococcus pentosaceus, Weissella cibaria, and Weissella confusa, and preferably selected from Fructilactobacillus sanfranciscensis, Furfurilactobacillus rossiae, Lactiplantibacillus plantarum.

32. The aid according to claim 29, wherein the microcapsules contain a combination of lactic acid bacteria belonging to the species Fructilactobacillus sanfranciscensis, Furfurilactobacillus rossiae and Lactiplantibacillus plantarum.

33. The aid according to claim 29, comprising a combination of microcapsules each containing lactic acid bacteria belonging to a species selected from Fructilactobacillus sanfranciscensis, Furfurilactobacillus rossiae, and Lactiplantibacillus plantarum.

34. The aid according to claim 29, wherein said food-grade carrier for baked food products comprises one or more of wheat flour, durum wheat flour, maize flour, rice flour, spelt flour, oat flour, pea flour, chickpea flour, soy flour, rye flour, millet flour, linseed flour, sesame seed flour, sunflower seed flour, gluten, maize starch, dried sourdough, brewer's yeast, salt, or mixtures thereof.

35. The aid according to claim 29, further comprising an effective amount of at least one added enzyme.

36. The aid according to claim 35, comprising an amount of at least one added enzyme equal to or higher than 0.01 and equal to or lower than 5 g/kg of the aid. 37 (New) The aid according to claim 35, wherein said at least one added enzyme is selected from xylanase, transglutaminase, cellulase, amylase, amyloglucosidase, lipase, phospholipase, asparaginase, oxidase of fungal or bacterial origin, and mixtures thereof. 38 (New) The aid according to claim 35, wherein said at least one added enzyme is of the hydrolysing and non-proteolytic type.

39. The aid according to claim 35, wherein said at least one added enzyme comprises a xylanase enzyme capable of hydrolysing arabinoxylans to produce xylose.

40. The aid according to claim 29, wherein said at least one gellable biopolymer is selected from starch or derivatives thereof, sodium alginate, calcium alginate, potassium alginate, ammonium alginate, gellan gum, xanthan gum, milk protein, gelatine, and mixtures thereof.

41. The aid according to claim 29, obtained by microencapsulation of viable lactic acid bacteria belonging to at least one species in a casing formed from a solution of gellable biopolymer and viable lactic acid bacteria, said solution comprising sodium alginate at a concentration equal to or higher than 2% and equal to or lower than 15% by weight.

42. The aid according to claim 41, wherein said sodium alginate has a viscosity equal to or higher than 4 and equal to or lower than 12 mPa·s when dissolved in a 1% by weight aqueous solution at a temperature of 20° C.

43. The aid according to claim 29, wherein said microcapsules have a relative humidity equal to or higher than 10% and equal to or lower than 17%.

44. The aid according to claim 29, wherein said microcapsules have a bulk density, evaluated on an uncompacted bulk mass of microcapsules, equal to or higher than 0.35 g/ml and equal to or lower than 0.55 g/ml.

45. The aid according to claim 29, wherein said microcapsules have an average size D10 equal to or higher than 200 μm and equal to or lower than 320 μm.

46. The aid according to claim 29, wherein said microcapsules have an average size D90 equal to or higher than 580 μm and equal to or lower than 1000 μm.

47. A process for the preparation of doughs for baked food products comprising mixing the aid according to claim 29 with water and a food-grade flour for baked food products.

48. The process according to claim 47, wherein the process is a straight dough preparation process.

49. A process for microencapsulating viable lactic acid bacteria in a casing including at least one gellable biopolymer, including:
a) providing a solution including a gellable biopolymer and viable lactic acid bacteria belonging to at least one species;
b) feeding drops of said solution of gellable biopolymer and viable lactic acid bacteria into a gelation bath including at least one gelling agent and allowing said drops to gel, and
c) drying the gelled droplets by air drying, obtaining microcapsules containing viable lactic acid bacteria belonging to at least one species, said microcapsules having an average size D50 equal to or higher than 350 μm and equal to or lower than 550 μm and a mechanical compressive strength, measured as set forth in the present description, equal to or higher than 3200 g and equal to or lower than 9000 g.

50. The process according to claim 49, wherein said step c) of drying the gelled droplets comprises subjecting the gelled droplets to a flow of air at a temperature equal to or higher than 25° and equal to or lower than 35° C. until said microcapsules reach a relative humidity equal to or higher than 10% and equal to or lower than 17%.

51. The process according to claim 49, wherein said at least one gellable biopolymer comprises sodium alginate having a viscosity equal to or higher than 4 and equal to or lower than 12 mPa—s when dissolved in a 1% by weight aqueous solution at a temperature of 20° C., and wherein a concentration of sodium alginate in said solution of gellable biopolymer and viable lactic acid bacteria is equal to or higher than 2% and equal to or lower than 15% by weight.

52. A process for producing an aid for the preparation of baked food products, comprising:
x) providing, by means of the process for microencapsulation according to claim 49, microcapsules containing viable lactic acid bacteria belonging to at least one species, enclosed in a casing including at least one gellable biopolymer, said microcapsules having an average size D50 equal to or higher than 350 μm and equal to or lower than 550 μm and a mechanical compressive strength, measured as set forth in the present description, equal to or higher than 3200 g and equal to or lower than 9000 g; and
y) dispersing said microcapsules in a quantity of a food-grade carrier for baked food products.

53. A dough for baked food products, comprising the aid according to claim 29, a food-grade flour for baked food products and water.

54. A process for preparing a baked food product, comprising:
forming a dough by mixing the aid according to claim 29, water and a food-grade flour for baked food products;
baking the dough in an oven.

55. The process for preparing a baked food product according to claim 54, wherein the process is a straight dough process for producing a baked food product.
